(19) United States
(12) Patent Application Publication
(10) Pub. No.: US 2023/0345900 A1
(43) Pub. Date: Nov. 2, 2023

(54) A TOMATO PLANT COMPRISING DOMINANT RESISTANCE GENES TO TOMATO BROWN RUGOSE FRUIT VIRUS

(71) Applicant: PHILOSEED LTD., Hazav (IL)

(72) Inventors: Ehud KOPELIOVITCH, Mazkeret Batya (IL); Irit GILAN, Kfar Harif (IL)

(21) Appl. No.: 18/016,959

(22) PCT Filed: Sep. 22, 2021
(86) PCT No.: PCT/IL2021/051147

§ 371 Date: Jan. 19, 2023

Related U.S. Application Data
(60) Provisional application No. 63/055,399, filed on Jul. 23, 2020.

(51) Int. Cl. A01H 6/82; A01H 1/00; C12Q 1/6895

(52) U.S. Cl. CPC A01H 6/825; A01H 1/126; C12Q 1/6895; C12Q 2600/13; C12Q 2600/158

(57) ABSTRACT

A tomato plant, harboring a partially dominant resistance gene or genes to tomato brown rugose fruit virus (ToBRFV). Such partially dominant resistance was obtained by crossing and back crossing of nine ToBRFV-resistant wild tomato plant lines, each harboring said partially dominant resistance gene or genes and a ToBRFV-susceptible cultivated Solanum lycopersicum. In addition, genetic markers for partially dominant resistance to tomato brown rugose fruit virus (ToBRFV) where identified and found to be related to chromosome 2, and genetic markers for sensitivity to tomato brown rugose fruit virus (ToBRFV) where identified and found to be related to chromosome 11.

### FIELD OF THE INVENTION

The present disclosure relates to the field of agriculture, more particularly to tomato plants harboring partially dominant resistance to the recently characterized Tobamovirus—tomato brown rugose fruit virus.

### BACKGROUND OF THE INVENTION

Tobamoviruses are a genus of plant viruses which pose a significant threat to vegetable and ornamental crops around the world, especially plants belonging to the Brassicaceae, Cucurbitaceae, Solanaceae and Malvaceae families. The impact of these viruses on crops can be economically devastating and result in considerable yield losses. Among these viruses are viruses which adversely infect tomato plants, the main ones being the tobacco mosaic virus (TMV), tomato mosaic virus (ToMV) tomato mild mottle virus (ToMMV), tobacco mild green mosaic virus (TMGMV) and pepper mild mottle virus (PMMoV).

Tobamoviruse genus is the largest genus (comprising 35 species) among the seven genera in the Virgaviridae family. These viruses are characterized by having a 300 nm long rod-shaped particle encapsulating a single-stranded, positive sense RNA genome of 6.2 to 6.4 kb encoding four proteins: the genomic segment expresses two replication-related proteins of 126 and 183 kDa, resulting from partial suppression of a stop codon; a 30-kDa movement protein (MP) is expressed through a sub-genomic RNA1 (sgRNA1); and a 17.5-kDa coat protein (CP) is expressed from a second sub-genomic RNA2 (sgRNA2).

In the spring of 2015 the occurrence of a new Tobamovirus in tomato (Solanum lycopersicum, cv. Candela) was discovered in greenhouses in Jordan (see “A new Tobamovirus infecting tomato crops in Jordan.” Salem et al; Arch Virol. 2016 February; 161 (2):503-6. Epub 2015 Nov. 19). This newly discovered virus was not shown to phylogenetically align with either ToMV or the TMV clades, but to stem from a branch leading to the TMV clade. The symptoms of the new virus included mild foliar symptoms at the end of the season, but strong brown rugose symptoms on the fruits. A later publication showed that the virus was also present in Israel in 2014, and it was established that the virus can also infect pepper (Capsicum annuum) plants (see “A new Israeli Tobamovirus isolate infects tomato plants harboring Tm-22 resistance genes.” Luria et al; PLoS ONE 2017).

Additionally, it was demonstrated that tomato plants harboring known resistance genes to Tobamoviruses exhibited susceptibility to the new virus (the virus was observed to break the resistance of the commonly used resistance genes against ToMV: Tm-1, Tm-2, and Tm-22). As the virus was clearly different from other known Tobamoviruses, it was given a new designation: Tomato brown rugose fruit virus (ToBRFV).

Symptoms appear to vary based on the affected variety. In some cases, severe brown rugose symptoms are present on almost all fruits, and in other instances, symptoms are mainly found on the vegetative parts in the form of severe or mild mosaic, necrosis, leaf distortion, or other symptoms. The severity of the viral symptoms on tomato fruits has a high impact on tomato growers, since this new viral disease results in fruits of very poor quality and value, which are almost unmarketable. The ToBRFV is easily transmitted by mechanical means, which facilitates its rapid spread, and makes it difficult to control. Transmission of the TOBRFV is also likely to occur through infected seeds.

Patent application WO2019110130A1 to Rijk Zwaan discloses a Solanum lycopersicum plant that is resistant to ToBRFV, said plant comprises a QTL on chromosome 11, and/or a QTL on chromosome 12, and/or a QTL on chromosome 6.

Patent application US20200048655A1 to SEMINIS VEGETABLE SEEDS discloses Tomato plants exhibiting resistance to Stemphylium. The invention also provides a Solanum lycopersicum plant comprising a recombinant chromosomal segment on chromosome 11, wherein said chromosomal segment comprises a Stemphylium resistance allele from Solanum pimpinellifolium conferring increased resistance to Stemphylium to said plant compared to a plant not comprising said allele, and wherein said recombinant chromosomal segment further comprises a Tomato Brown Rugose Fruit Virus (ToBRFV) resistance allele. In some embodiments, said ToBRFV resistance allele is located within a chromosomal segment flanked by marker locus M1 and marker locus M3 on chromosome 11 in said plant. In other embodiments, said plant is homozygous for said ToBRFV resistance allele.

Patent application US20200077614A1 to VILMORIN & CIE discloses Solanum lycopersicum plant resistant to Tomato Brown Rugose Fruit virus comprising in its genome the combination of the Tm-1 resistance gene on chromosome 2, and at least one quantitative trait locus (QTL) chosen from QTL3 on chromosome 11, QTL1 on chromosome 6 and QTL2 on chromosome 9, that independently confer to the plant foliar and/or fruit tolerance to ToBRFV.

Patent application WO2020148021 to Enza Zaden Beheer B. V. discloses a plant of the S. lycopersicum species that is resistant to Tobamovirus, wherein the plant comprises one or more genomic sequences or locus providing said resistance.

In view of the prior art documents and given the various challenges faced by tomato growers around the world, there is still an unmet long-felt need to obtain tomato varieties which are resistant in a dominant manner to tomato brown rugose fruit virus.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention.

FIG. 1 depicting symptomless tomato plants exposed to ToBRFV, which are served to search resistant varieties according to the present invention;

FIG. 2 depicting necrosis reaction on N. glutinosa leaves from symptomless tomato plants exposed to ToBRFV.

FIG. 3 depicting a flowchart detailing a crossing program used for generating the resistant plants of the present application;

FIG. 4 depicting a flowchart detailing a crossing program used for generating the resistant plants of the present application;

FIG. 5 depicting the ToBRFV-resistant tomato leaf of the present invention (FIG. 5A) compared to a ToBRFV-sensitive tomato leaf (FIG. 5B);

FIG. 6 depicting the ToBRFV-resistant tomato fruit of the present invention (left) compared to a ToBRFV-sensitive tomato fruit (right); and

FIG. 7 depicting the ToBRFV-resistant tomato plant of the present invention (FIG. 7A) compared to a ToBRFV-sensitive tomato plant (FIG. 7B).

FIG. 8 depicting cross the parental lines samples sequencing data coverage.

FIG. 9 depicting cross the population-1 20-28 samples sequencing data coverage.

FIG. 10 depicting population 2, sequencing data coverage cross samples.

FIG. 11 depicting the correlation between ToBRFV ELISA test and Nicotina glutinosa Bioassay.

FIG. 12 depicting the LOD scores at each contig for each of mapping population-1.

FIG. 13 depicting the LOD scores at each contig for each of mapping population-2.

### SUMMARY OF THE INVENTION

It is one object of the present invention to disclose a tomato plant harboring a partially dominant resistance gene/genes to tomato brown rugose fruit virus (ToBRFV).

It is another object of the present invention to disclose the tomato plant as described above, wherein said plant is the offspring of crossing a ToBRFV-resistant wild tomato plant with a ToBRFV-susceptible cultivated tomato plant or a ToBRFV-resistant wild tomato plant with a ToBRFV-susceptible wild tomato plant.

It is another object of the present invention to disclose the tomato plant as described above, wherein said ToBRFV-resistant wild tomato plant harbors said partially dominant resistance gene/genes.

It is another object of the present invention to disclose the tomato plant as described above, wherein said ToBRFV-resistant wild tomato plant is selected from a group consisting of LA0107 (S. corneliomulleri), LA0361, LA1918, LA2650 (S. habrochaites), LA1938, LA1969, LA2748, LA2755 and LA2931 (S. chilense).

It is another object of the present invention to disclose the tomato plant as described above, wherein said ToBRFV-susceptible cultivated tomato plant is Solanum lycopersicum.

It is another object of the present invention to disclose genetic markers for the use in identification or selection of tomato plants harboring dominant resistance to tomato brown rugose fruit virus (ToBRFV).

It is another object of the present invention to disclose the genetic markers as described above, wherein said markers are configured to be linked to the gene or genes or QTL conferring partially dominant resistance to ToBRFV in said tomato plants.

It is another object of the present invention to disclose the genetic markers as described above, wherein said gene or genes or QTL is on chromosome 2.

It is another object of the present invention to disclose the genetic markers as described above, wherein said gene, or genes, or QTL on chromosome 2 comprises at least one genomic sequences sequence selected from a group under key number and position as indicated in Appendix 3:

| | | |
|---|---|---|
| BC1_2_L2_15031000 | 15031000, BC1_2_L2_18820000 | 18820000, |
| BC1_2_L2_15795000 | 15795000, BC1_2_L3_29720000 | 29720000, |
| BC1_2_L3_22563000 | 22563000, BC1_2_L3_25275000 | 25275000, |
| BC1_2_L3_25187000 | 25187000, BC1_2_L3_29656000 | 29656000, |
| BC1_2_L3_24977000 | 24977000, BC1_2_L3_24955000 | 24955000, |
| BC1_2_L3_27949000 | 27949000, BC1_2_L3_23415000 | 23415000, |
| BC1_2_L3_28862000 | 28862000, BC1_2_L3_26512000 | 26512000, |
| BC1_2_L3_24581000 | 24581000, BC1_2_L4_32405000 | 32405000, |
| BC1_2_L4_33723000 | 33723000, BC1_2_L4_33383000 | 33383000, |
| BC1_2_L4_32180000 | 32180000, BC1_2_L4_33110000 | 33110000, |
| BC1_2_L4_32404000 | 32404000, BC1_2_L4_34990000 | 34990000, |
| BC1_2_L4_35114000 | 35114000, BC1_2_L4_33583000 | 33583000, |
| BC1_2_L4_35860000 | 35860000, BC1_2_L4_32796000 | 32796000. |

It is another object of the present invention to disclose genetic markers for the use in identification and/or negative selection of tomato plants harboring sensitivity to tomato brown rugose fruit virus (ToBRFV).

It is another object of the present invention to disclose the genetic markers as described above, wherein said markers are configured to be linked to the gene or genes or QTL conferring sensitivity to ToBRFV in said tomato plants.

It is another object of the present invention to disclose the genetic markers as described above, wherein said gene or genes or QTL is on chromosome 11.

It is another object of the present invention to disclose the genetic markers as described above, wherein said gene or genes or QTL on chromosome 11 comprises at least one genomic sequences sequence selected from a group under key number and position as indicated in Appendix 3:

| | | |
|---|---|---|
| BC0_11_L1_10826000 | 10826000, BC0_11_L1_9649000 | 9649000, |
| BC0_11_L1_10837000 | 10837000, BC0_11_L1_10827000 | 10827000, |
| BC0_11_L1_10141000 | 10141000, BC0_11_L2_11459000 | 11459000, |
| BC0_11_L2_12276000 | 12276000, BC0_11_L2_12706000 | 12706000. |

It is another object of the present invention to disclose a method for identifying and/or selecting a tomato plant, or tomato seed, or tomato plant tissue, or tomato plant cell, harboring a partially dominant resistance gene or genes to tomato brown rugose fruit virus (ToBRFV) comprising steps:
1. obtaining a tomato plant, or tomato seed, or tomato plant tissue, or tomato plant cell,
2. identifying said genetic markers as described above.

It is another object of the present invention to disclose a method for identifying and/or selecting against a tomato plant, or tomato seed, or tomato plant tissue, or tomato plant cell, harboring a sensitivity gene or genes to tomato brown rugose fruit virus (ToBRFV) comprising steps:
1. obtaining a tomato plant, or tomato seed, or tomato plant tissue, or tomato plant cell,
2. identifying said genetic markers as described above.

It is another object of the present invention to disclose a method for identifying and/or selecting a tomato plant, or tomato seed, or tomato plant tissue, or tomato plant cell, harboring a partially dominant resistance gene or genes or QTL to tomato brown rugose fruit virus (ToBRFV), and for identifying and/or selecting against a sensitivity gene or genes or QTL to tomato brown rugose fruit virus (ToBRFV) comprising steps:
1. obtaining a tomato plant, or tomato seed, or tomato plant tissue, or tomato plant cell,
2. identifying said genetic markers as described above,
3. selecting the tomato plant, or tomato seed, or tomato plant tissue, or tomato plant cell, harboring said genetic markers as described above,
4. identifying said genetic markers as described above in the selected plants of the above section 3,
5. selecting against the tomato plant, or tomato seed, or tomato plant tissue, or tomato plant cell, harboring said genetic markers as described above,
6. obtaining a tomato plant, or tomato seed, or tomato plant tissue, or tomato plant cell harboring a partially dominant resistance gene or genes, or QTL, and lacking a sensitivity gene or genes, or QTL to tomato brown rugose fruit virus (ToBRFV).

It is another object of the present invention to disclose a method for identifying and/or selecting a tomato plant, or tomato seed, or tomato plant tissue, or tomato plant cell, harboring a partially dominant resistance gene or genes or QTL to tomato brown rugose fruit virus (ToBRFV), and for identifying and/or selecting against a sensitivity gene or genes or QTL to tomato brown rugose fruit virus (ToBRFV) comprising steps:
1. obtaining a tomato plant, or tomato seed, or tomato plant tissue, or tomato plant cell,
2. identifying said genetic markers for sensitivity to tomato brown rugose fruit virus (ToBRFV) as described above,
3. selecting against the tomato plant, or tomato seed, or tomato plant tissue, or tomato plant cell, harboring said genetic markers as described above,
4. identifying partially dominant resistance to tomato brown rugose fruit virus (ToBRFV) genetic markers as described above,
5. selecting the tomato plant, or tomato seed, or tomato plant tissue, or tomato plant cell, harboring said partially dominant resistance to tomato brown rugose fruit virus (ToBRFV) genetic markers as described above,
6. obtaining a tomato plant, or tomato seed, or tomato plant tissue, or tomato plant cell harboring a partially dominant resistance gene or genes, or QTL, and lacking a sensitivity gene or genes, or QTL to tomato brown rugose fruit virus (ToBRFV).

It is another object of the present invention to disclose a tomato plant harboring a partially dominant resistance gene or genes and lacking sensitivity to tomato brown rugose fruit virus (ToBRFV).

It is another object of the present invention to disclose the tomato plant as described above, wherein said plant is the offspring of crossing a ToBRFV-resistant wild tomato plant, which also lack sensitivity to ToBRFV, with a ToBRFV-susceptible cultivated tomato plant or a TOBRFV-resistant wild tomato plant with a ToBRFV-susceptible wild tomato plant.

It is another object of the present invention to disclose the tomato plant as described above, wherein said ToBRFV-resistant wild tomato plant harbors said partially dominant resistance gene or genes and lacks said sensitivity genes.

It is another object of the present invention to disclose the tomato plant as described above, wherein said partially dominant resistance gene or genes is on chromosome 2, said sensitivity gene or genes is on chromosome 11.

It is another object of the present invention to disclose genetic markers for the use in identification or selection of tomato plants harboring partially dominant resistance and lacking sensitivity to tomato brown rugose fruit virus (ToBRFV).

It is another object of the present invention to disclose the genetic markers as described above, wherein said markers are configured to be linked to the gene or genes or QTL conferring partially dominant resistance to ToBRFV and sensitivity in said tomato plants.

It is another object of the present invention to disclose the genetic markers as described above wherein said gene or genes or QTL conferring partially dominant resistance to ToBRFV is on chromosome 2 and wherein said gene or genes or QTL conferring sensitivity to ToBRFV is on chromosome 11.

It is another object of the present invention to disclose the genetic markers as described above, wherein said gene, or genes, or QTL conferring partially dominant resistance to ToBRFV on chromosome 2 comprises at least one genomic sequences sequence selected from a group under key number and position as indicated in Appendix 3:

| | | |
|---|---|---|
| BC1_2_L2_15031000 | 15031000, BC1_2_L2_18820000 | 18820000, |
| BC1_2_L2_15795000 | 15795000, BC1_2_L3_29720000 | 29720000, |
| BC1_2_L3_22563000 | 22563000, BC1_2_L3_25275000 | 25275000, |
| BC1_2_L3_25187000 | 25187000, BC1_2_L3_29656000 | 29656000, |
| BC1_2_L3_24977000 | 24977000, BC1_2_L3_24955000 | 24955000, |
| BC1_2_L3_27949000 | 27949000, BC1_2_L3_23415000 | 23415000, |
| BC1_2_L3_28862000 | 28862000, BC1_2_L3_26512000 | 26512000, |
| BC1_2_L3_24581000 | 24581000, BC1_2_L4_32405000 | 32405000, |
| BC1_2_L4_33723000 | 33723000, BC1_2_L4_33383000 | 33383000, |
| BC1_2_L4_32180000 | 32180000, BC1_2_L4_33110000 | 33110000, |
| BC1_2_L4_32404000 | 32404000, BC1_2_L4_34990000 | 34990000, |
| BC1_2_L4_35114000 | 35114000, BC1_2_L4_33583000 | 33583000, |
| BC1_2_L4_35860000 | 35860000, BC1_2_L4_32796000 | 32796000; |

and wherein said gene, or genes, or QTL conferring sensitivity to ToBRFV on chromosome 11 comprises at least one genomic sequences sequence selected from a group under key number and position as indicated in Appendix 3:

| | |
|---|---|
| BC0_11_L1_10826000 10826000, | |
| BC0_11_L1_96490009649000, | BC0_11_L1_10837000 10837000, |
| BC0_11_L1_10827000 10827000, | BC0_11_L1_10141000 10141000, |
| BC0_11_L2_11459000 11459000, | BC0_11_L2_12276000 12276000, |
| BC0_11_L2_12706000 12706000. | |

It is another object of the present invention to disclose the tomato plant as described above, wherein said ToBRFV-resistant wild tomato plant, which also lack sensitivity to ToBRFV, is selected from a group consisting of LA0107 (S. corneliomulleri), LA0361, LA1918, LA2650 (S. habrochaites), LA1938, LA1969, LA2748, LA2755 and LA2931 (S. chilense).

It is another object of the present invention to disclose the tomato plant as described above, wherein said ToBRFV-susceptible cultivated tomato plant is Solanum lycopersicum.

It is another object of the present invention to disclose a seed of the tomato plant as described above.

It is another object of the present invention to disclose propagation materials of the tomato plant as described above.

### DETAILED DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a tomato plant which is resistant to tomato brown rugose fruit virus (ToBRFV) in a partially dominant manner.

The present invention relates to a tomato plant, wherein said ToBRFV resistance gene and/or one or more genomic sequences are as found in the deposit accession number NCIMB 43817. NCIMB 43817. L. esculentum x L. habrochaites lycopersicum seeds were deposited on 16 Jul. 2021 at NCIMB Ltd, Ferguson Building, Craibstone Estate Bucksburn, AB21 9YA Aberdeen, United Kingdom.

As used herein after, the term “about” refers to any value being up to 25% lower or greater than the defined measure.

As used herein after, the term “Tobamovirus” refers to a genus of plant viruses (comprising 35 species) belonging to the Virgaviridae family. These viruses are characterized by having a 300 nm long rod-shaped particle encapsulating a single-stranded, positive sense RNA genome of 6.2 to 6.4 kb encoding four proteins: the genomic segment expresses two replication-related proteins of 126 and 183 kDa, resulting from partial suppression of a stop codon; a 30-kDa movement protein (MP) is expressed through a sub-genomic RNA1 (sgRNA1); and a 17.5-kDa coat protein (CP) is expressed from a second sub-genomic RNA2 (sgRNA2). The Tobamoviruses infect mostly plants belonging to the Brassicaceae, Cucurbitaceae, Solanaceae and Malvaceae families. The most known Tobamoviruses which infect tomato plants are: the tobacco mosaic virus (TMV), tomato mosaic virus (ToMV) and tomato mild mottle virus (ToMMV), tobacco mild green mosaic virus (TMGMV) and pepper mild mottle virus (PMMoV).

As used herein after, the term “Tomato brown rugose fruit virus (ToBRFV)” refers to a recently discovered virus, belonging to the Tobamoviruses genus. This virus was first discovered in the spring of 2015 in Jordan, and it was also found in Israel, Turkey, the Netherlands, Mexico, and USA. The genome of the virus comprises 6393-nt single-stranded RNA (ssRNA) encoding four proteins. The ToBRFV causes the following symptoms: severe brown rugose symptoms on fruits, yellow spots on fruits, severe or mild mosaic on leaves, leaf narrowing, necrosis, leaf distortion and more. The ToBRFV is easily transmitted by mechanical means, and through infected seeds.

As used herein after, the term “variety” or “varieties” refers to the usual denomination in agricultural industry and correspond to a plant of a given botanical taxon which is distinct from other existing plant, which is uniform and stable.

According to the international seed federation, “susceptibility” is the inability of a plant variety to restrict the growth and/or development of a specified pest. “Resistance” is the ability of a plant variety to restrict the growth and/or development of a specified pest and/or the damage it causes when compared to susceptible plant varieties under similar environmental conditions and pest pressure. Resistant varieties may exhibit some disease symptoms or damage under heavy pest pressure. Two levels of resistance are defined. “High resistance (HR)”: plant varieties that highly restrict the growth and/or development of the specified pest and/or the damage it causes under normal pest pressure when compared to susceptible varieties. These plant varieties may, however, exhibit some symptoms or damage under heavy pest pressure. “Intermediate resistance (IR)”: plant varieties that restrict the growth and/or development of the specified pest and/or the damage it causes but may exhibit a greater range of symptoms or damage compared to high resistant varieties. Intermediate resistant plant varieties will still show less severe symptoms or damage than susceptible plant varieties when grown under similar environmental conditions and/or pest pressure.

As used herein after, the term “partially dominant resistance” refers to inheritance of a dominant gene or genes from one parent, which confers partial resistance in the offspring to an infection.

As used herein, the term “homozygous” refers to a genetic condition or configuration existing when two identical or like alleles reside at a specific locus, but are positioned individually on corresponding pairs of homologous chromosomes in the cell of a diploid organism.

Conversely, as used herein, the term “heterozygous” means a genetic condition or configuration existing when two different or unlike alleles reside at a specific locus, but are positioned individually on corresponding pairs of homologous chromosomes in the cell of a diploid organism.

The term “selfing” used herein refers in some embodiments to the production of seed by self-fertilization or self-pollination; i.e., pollen and ovule are from the same plant/parental line.

The term “phenotype” is understood within the scope of the invention to refer to a distinguishable characteristic(s) of a genetically controlled trait.

As used herein, the phrase “phenotypic trait” refers to the appearance or other detectable characteristic of an individual, resulting from the interaction of its genome, proteome and/or metabolome with the environment.

As used herein after, the term “Nicotiana glutinosa assay” refers to an assay designed for the detection of a plant virus. The assay comprises the inoculation of the indicator plant Nicotiana glutinosa leaves with extracts (such as sap) from infected plants. If the infected plants are resistant to the virus, the N. glutinosa leaves are not expected to develop any reaction to the virus. If the infected plants are sensitive to the virus, their extracts contain significant amounts of viral particles, and in contact with the N. glutinosa leaves, the leaves will necrotize.

As used herein after, the term “quantitative trait locus (QTL)” refers to a part of the DNA which correlates with a variation of a quantitative trait in the phenotype of a population of organisms. QTLs are mapped by identifying which molecular markers correlate with an observed trait.

As used herein after, the term “K-mer” refers to a substrings of length k contained within a biological sequence. In bioinformatics, k-mers are composed of nucleotides and primarily used as a tool in computational genomics, sequence analysis, and DNA assembly. The frequency of a set of k-mers in a species' genome, or in a genomic region, or in a class of sequences can be used as a “finger print” of the underlying sequence. Comparing these frequencies is computationally advantageous compared to sequence alignment and is an important method in alignment-free sequence analysis.

As used herein after, the term “Basic Local Alignment Search Tool (BLAST)” refers to a computational search program configured to find regions of local similarity between sequences. The BLAST program compares nucleotide or protein sequences to sequence databases and calculates the statistical significance of matches. BLAST can be used to infer functional and evolutionary relationships between sequences as well as help identify members of gene families (blast.ncbi.nlm.nih.gov/Blast.cgi).

As used herein after, the term “logarithm of the odds score (LOD score)” refers to a statistical numerical estimate of whether two genetic loci are (two genes, or a gene and a disease) physically close enough to each other on a particular chromosome that they are likely to be inherited together. A LOD score of 3 or higher is generally understood to mean that two genes are located close to each other on the chromosome and are most likely linked. In terms of significance, a LOD score of 3 means the odds are 1,000:1 that the two genes are linked and therefore inherited together.

As used herein after, the term “p-value” refers to a measure of the probability that an observed difference could have occurred just by random chance according to the null hypothesis significance testing. The lower the p-value, the greater the statistical significance of the observed difference. P-value can be used as an alternative to or in addition to pre-selected confidence levels for hypothesis testing.

As used herein after, the term “Next Generation Sequencing (NGS)” refers to a massively parallel sequencing technology. NGS is used to determine the order of nucleotides in entire genomes or targeted regions of DNA or RNA. DNA polymerase catalyzes the incorporation of fluorescently labeled deoxyribonucleotide triphosphates (dNTPs) into a DNA template strand during sequential cycles of DNA synthesis. During each cycle, at the point of incorporation, the nucleotides are identified by fluorophore excitation. The major advantage of the NGS us that instead of sequencing a single DNA fragment, NGS extends this process across millions of fragments in a massively parallel fashion. NGS comprises of four basic steps:
1. Library Preparation—The sequencing library is prepared by random fragmentation of the DNA or cDNA sample, followed by5′ and 3′ adapter ligation. Alternatively, “tag mentation” combines the fragmentation and ligation reactions into a single step that greatly increases the efficiency of the library preparation process.
2. Cluster Generation—For cluster generation, the library is loaded into a flow cell where fragments are captured on a lawn of surface-bound oligos complementary to the library adapters. Each fragment is then amplified into distinct, clonal clusters through bridge amplification.
3. Sequencing—reversible terminator—based method that detects single bases as they are incorporated into DNA template strands.
4. Data Analysis—During data analysis and alignment, the newly identified sequence reads are aligned to a reference genome. Following alignment, many variations of analysis are possible, such as single nucleotide polymorphism (SNP) or insertion-deletion (indel) identification, read counting for RNA methods, phylogenetic or metagenomic analysis, etc.

As used herein after, the term “skim sequencing” refers to low coverage whole-genome sequencing used for the identification of large numbers of polymorphic markers. Skim sequencing is being extensively used for genotyping in diverse plant species and has a wide range of applications, particularly in quantitative trait loci (QTL) mapping, genome wide association studies (GWAS), fine genetic map construction, and identification of recombination and gene conversion events in various breeding programs. The skim sequencing approaches are based on hybridization methods using developed (known) bait sequences, transcriptome sequencing, and reduced representation methods. By contrast, genome skimming is by far one of the simplest methodologies, involving random sampling of a small percentage of total gDNA. This approach has been used successfully at varying taxonomic levels, for intraspecific ‘ultra-barcoding’, intergeneric and family-wide phylogenomic analyses

As used herein after, the term “Contig” refers to a physical DNA map or a consensus region of DNA of contiguous sequence generated by overlapping series of sequence reads. A common approach to obtain sequence contig is the “bottom-up sequencing” which involves shearing genomic DNA into many small fragments (“bottom”), sequencing these fragments, reassembling them back into contigs and eventually the entire genome (“up”). A contig contains no gaps.

As used herein after, the term “The likelihood ratio test (LR test)” refers to a statistical test performed by estimating two models and comparing the fit of one model to the fit of the other. To test whether the observed difference in model fit is statistically significant, the LR test comparing the log likelihoods (The likelihood is the probability the data given the parameter estimates) of the two models, if this difference is statistically significant, then the less restrictive model (the model with more variables) is said to fit the data significantly better than the more restrictive model. If one has the log likelihoods from the models, the LR test is fairly easy to calculate. The formula for the LR test statistic is:

$$L R = - 2 ln ( \frac{L ( {m}_{1} )}{L ( {m}_{2} )} ) = 2 ( log lik ( {m}_{2} ) - log lik ( {m}_{1} ) )$$

Where L(m_{*}) denotes the likelihood of the respective model (either Model 1 or Model₂), and loglik (m_{*}*) the natural log of the model's final likelihood (the log likelihood). Where m₁ is the more restrictive model, and m2 is the less restrictive model. The resulting test statistic is distributed chi-squared, with degrees of freedom equal to the number of parameters that are constrained.

As used herein after, the term “False Discovery Rates (FDR)” refers to the expected number of false positives out of all hypothesis tests conducted. When conducting and analyzing results of multiple comparisons for example, genome wide studies, each test is a “feature”, often thousands of hypothesis tests, “features” are conducted simultaneously. In a multiple comparison there is an increased probability of false positives. The more features, the higher the chances of a null feature being called significant. The false positive rate (FPR), is the expected number of false positives out of all hypothesis tests conducted. In order to be able to identify as many significant comparisons as possible while still maintaining a low false positive rate, the False Discovery Rate (FDR) and its analog the q-value are utilized. Controlling for the false discovery rate (FDR) is a way to identify as many significant features as possible while incurring a relatively low proportion of false positives. The FDR is the rate that features called significant are truly null.

FDR=expected (# false predictions/# total predictions). The power of the FDR method is uniformly larger than Bonferroni methods. The power advantage of the FDR over the Bonferroni methods increases with an increasing number of hypothesis tests.

As used herein after, the term “Enzyme Linked Immunosorbent Assay a powerful method for detecting and quantifying a specific protein in a complex mixture. The method enables analysis of protein samples immobilized in microplate wells, which passively bind antibodies and proteins, using specific antibodies. It is this binding and immobilization of reagents that makes ELISAs easy to design and perform. Having the reactants of the ELISA immobilized to the microplate surface makes it easy to separate bound from non-bound material during the assay. This ability to use high-affinity antibodies and wash away non-specific bound materials makes ELISA a powerful tool for measuring specific analytes within a crude preparation.

Although many variants of ELISA have been developed and used in different situations, they all depend on the same basic elements:
1. Coating/capture—direct or indirect immobilization of antigens to the surface of polystyrene microplate wells.
2. Plate blocking—addition of irrelevant protein or other molecule to cover all unsaturated surface-binding sites of the microplate wells.
3. Probing/detection—incubation with antigen-specific antibodies that affinity-bind to the antigens.
4. Signal measurement—detection of the signal generated via the direct or secondary tag on the specific antibody.

The most commonly used enzyme labels are horseradish peroxidase (HRP) and alkaline phosphatase (AP). A large selection of substrates is available commercially for performing ELISA with an HRP or AP conjugate. The choice of substrate depends upon the required assay sensitivity and the instrumentation available for signal-detection (spectrophotometer, fluorometer, or luminometer).

There are several formats used for ELISAs: direct, indirect, sandwich or competitive capture and detection methods. The key step is immobilization of the antigen of interest, accomplished by either direct adsorption to the assay plate or indirectly via a capture antibody that has been attached to the plate. The antigen is then detected either directly (labeled primary antibody) or indirectly (such as labeled secondary antibody). The most widely used ELISA assay format is the sandwich ELISA assay, which indirectly immobilizes and indirectly detects the presence of the target antigen. This type of capture assay is called a “sandwich” assay because the analyte to be measured is bound between two primary antibodies, each detecting a different epitope of the antigen—the capture antibody and the detection antibody. The sandwich ELISA format is highly used because of its sensitivity and specificity. Competitive ELISA is a strategy that is commonly used when the antigen is small and has only one epitope or antibody binding site. One variation of this method consists of labeling purified antigen instead of the antibody. Unlabeled antigen from samples and the labeled antigen compete for binding to the capture antibody. A decrease in signal from the purified antigen indicates the presence of the antigen in samples when compared to assay wells with labeled antigen alone.

The present invention provides tomato plants which exhibit resistance to the tomato brown rugose fruit virus (ToBRFV) in a partially dominant manner.

Plants can be targeted and infected by a variety of pathogens (bacteria, viruses, fungi etc.), and as a result, defense mechanisms conferring resistance have evolved. In general, those mechanisms comprise genes that sense the presence of a specific pathogen by recognizing specific components of that pathogen, referred to as “avirulence factors”. Triggering of the resistance genes can lead to defense pathways, such as the hypersensitive response, where the infected areas undergo programmed cell death and necrotic lesions aiming to eliminate the pathogen are observed. In some cases, the resistance mechanism is so intensive, that no lesions are apparent.

The resistance can be dominant or recessive (where all resistance gene copies are in dominant or recessive state, respectively.) In order for the progeny to inherit dominance resistance, it suffices that only one of the parental plants carries the dominant resistance gene/genes. Using a plant with dominant resistance to cross a susceptible plant facilitates the insertion of the dominance genes to elite hybrids.

It is also noteworthy that wild species used for production of resistant offspring by crosses often pass down undesired or disadvantageous traits which might manifest in the homozygote state as recessive traits. The present invention discloses ToBRFV-resistant tomato plants, wherein the resistance is partially dominant. This partially dominant resistance is inherited solely from one of the parental lines used to generate to tomato plants of the present invention, since one of the parental lines is a wild tomato species possessing said dominant resistance.

In a preferred embodiment of the present invention, the ToBRFV resistant tomato plants are produced by crossing a resistant wild tomato parent harboring a dominant resistance gene or genes with a susceptible cultivated tomato plant (Solanum lycopersicum).

In yet another preferred embodiment of the present invention, the ToBRFV resistant parent used for said cross is the male parent, whose pollen grains are utilized to fertilize the cultivated tomato female plant.

In yet another preferred embodiment of the present invention, the ToBRFV resistant parent used for said cross is the female parent, whose ovules are fertilized with pollen of the male parent.

In yet another preferred embodiment of the present invention, the progeny of said cross is symptomless when exposed to ToBRFV.

In yet another preferred embodiment of the present invention, the resistance to ToBRFV is passed down through the offspring of said cross via partially dominant resistance gene/genes.

In yet another preferred embodiment of the present invention, the dominant resistance to ToBRFV in cultivated lines of S. lycopersicum, could use to create ToBRFV resistant hybrid varieties.

In an additional preferred embodiment of the present invention, a ToBRFV resistant tomato plant is obtained by crossing a TOBRFV resistant wild tomato plant with a susceptible wild tomato plant.

In an additional preferred embodiment of the present invention, a ToBRFV resistant tomato plant is obtained by crossing a ToBRFV resistant hybrid with a resistant wild tomato plant.

### EXAMPLE 1

An extensive screening of about 50 wild tomato species accessions, obtained from the tomato genetics resource center (TGRC) Gene bank at UC Davis, resulted in the identification of 9 accessions, exhibiting some forms of resistance to ToBRFV. The determination of resistance was carried out using the bioassay method which comprises: (i) lack of phenotypic symptoms after actively infecting the plants with the virus and (ii) no symptoms on Nicotiana glutinosa plants that were infected with the plant's sap.

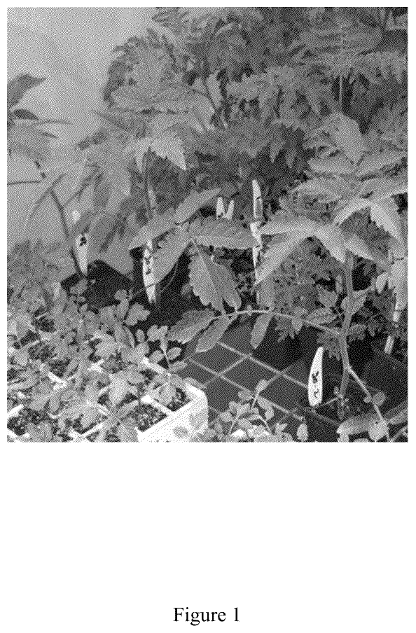

Reference is now made to FIG. 1 depicting tomato plants which did not manifest any symptoms after being actively infected with ToBRFV. These symptomless plants are further examined in the Nicotiana glutinosa assay, during which the sap of these plants comes in contact with leaves of Nicotiana glutinosa to monitor existence of necrosis in response to the viral infection.

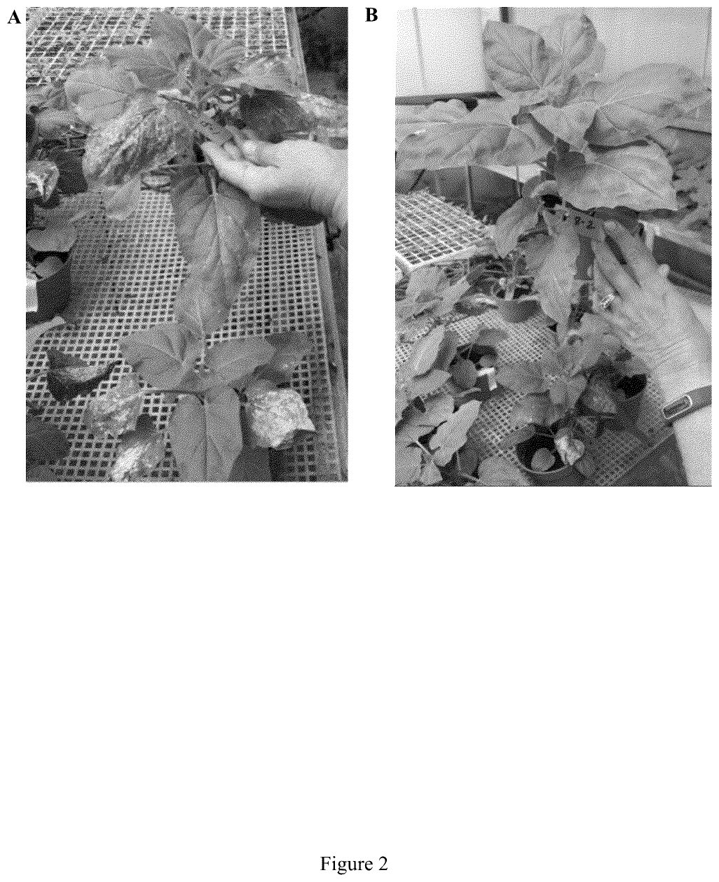

Reference is also made to FIG. 2 depicting Nicotiana glutinosa leaves which exhibit necrosis (FIG. 2A), and leaves which do not exhibit any signs of necrosis (FIG. 2B) after being exposed to sap from TOBRFV-infected symptomless tomato plants.

### EXAMPLE 2

Plants from each of the inoculated accessions which exhibited resistance to the ToBRFV and successfully passed the bioassays described in example 1 of the detailed description, were grown in a greenhouse to multiply and were used as pollen source for crosses after retesting for absence of virus. Plants that manifested late symptoms or had any positive response in the N. glutinosa assay were eliminated. The resistant plants were derived from the following 9 accessions: LA0107 (S. corneliomulleri), LA0361, LA1918, LA2650 (S. habrochaites), LA1938, LA1969, LA2748, LA2755 and LA2931 (S. chilense).

A research program was set up to determine if the resistance could be transferred to Solanum lycopersicum, and to identify the genetics underlying the ToBRFV resistance within the different accessions. One obstacle to overcome is that not all wild species cross easily with S. lycopersicum to set fruit that contain fertile seeds (even if seeds are created, they might not germinate because of inherent incompatibility).

### EXAMPLE 3

As mentioned above, to investigate the genetic basis of Tomato brown rugose fruit virus (ToBRFV), the inventors screened about 50 wild tomato sources including S. arcanum, S. chilense, S. corneliomulleri, S. habrochaites, S. huaylasense, S. pennellii and S. peruvianum.

### The Mapping Populations Design and Phenotype

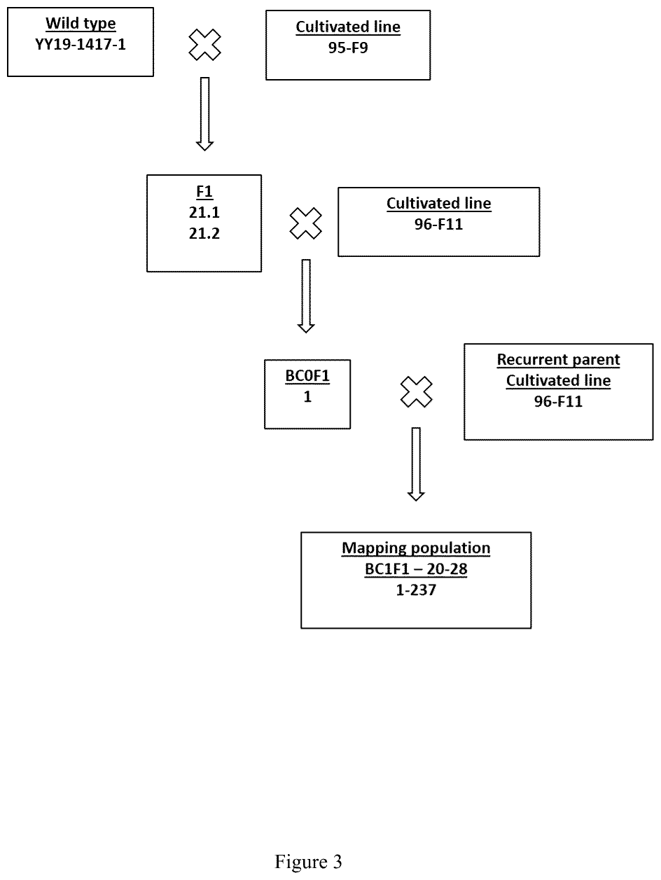

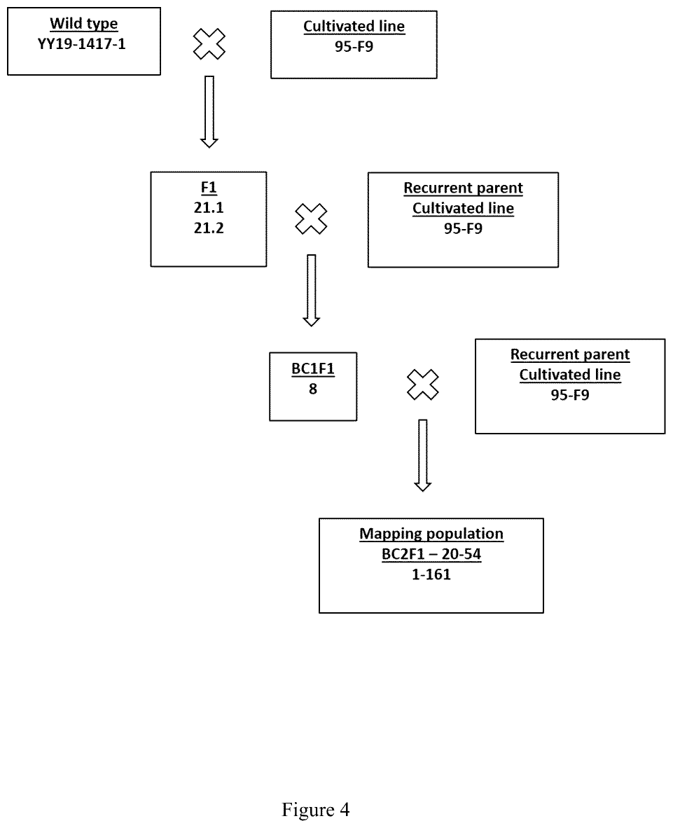

The following crossing programs are presented in FIG. 3 and FIG. 4 and elaborated in the following disclosure: crossing between the line YY19-1417-1 (S. habrochaites) with one of Philoseed parental line 95-F9 show high resistance (HR) to ToBRFV. This phenotype indicates a partially dominant mode of inheritance (see FIG. 3 and FIG. 4).

The F1 plants were crossed with different esculentum backgrounds representing Philoseed parental lines to produce BC0F1 and BC1F1 seeds. The BC0F1 plants show segregating phenotype, as well as, on generations BC1F1, BC1F2 and BC2F1.

The inventors used two mapping populations 20-28 (FIG. 3) and 20-54 (FIG. 4) which includes 237 BC1F1 and 161 BC2F1 plants, respectively (see Table 1 and Table 2).

The first population (20-28) was generated by crossing the F1 plants with the parental line 96-F11 (indeterminate beef type tomato) generating BC0F1 and then backcrossing a single (HR)BC0F1 plant with its recurrent line 96-F11 to produce the BC1F1 mapping population (see Table 3 below). The second population (20-54) was generated by crossing the F1 plants with the parental line 95-F9 (indeterminate cluster type tomato) generating BC1F1 and then backcrossing a single (HR) BC1F1 plant with its recurrent line 95-F9 to produce the BC2F1 mapping population (see Table 3).

It is important to mention here that population 20-28 is genetically BC2F1 and not BC1F1 since the initial cross was done on line 95-F9 and the Backcross was done on different line (96-F11) (see FIG. 3 and FIG. 4), therefore, the ratio of the wild background in the mapping populations is accordingly, about 12.5%.

### The Sampling for Mapping the Trait

Leaf samples from single plants of the 4 parents of the mapping population (two of each, BC0F1-1+96-F11, and BC1F1-8+95-F9, respectively) were sent for DNA extraction and whole genome sequencing by NRGene Ltd (see Table 4, Table 5 and Appendix 1) The leaf samples of all the plants from the two mapping populations were sent for DNA extraction and genotyping (see Table 4, Table 5 and Appendix 1).

TABLE 5
DNA extraction results of population 20-54 samples.

| | | | | UV | | |
|---|---|---|---|---|---|---|
| Master | Master | | | concen- | | |
| plate | plate | | | tration | | |
| name | barcode | Well | Sample ID | (in ng/μL) | 260/280 | 260/230 |
| 2112-mp3 | TSK22014 | A01 | 196 | 7.90 | 1.71 | 3.81 |
| 2112-mp3 | TSK22014 | B01 | 199 | 11.40 | 1.74 | 1.67 |
| 2112-mp3 | TSK22014 | C01 | 201 | 19.72 | 1.74 | 1.98 |
| 2112-mp3 | TSK22014 | D01 | 202 | 10.81 | 1.70 | 3.42 |
| 2112-mp3 | TSK22014 | E01 | 204 | 11.06 | 1.74 | 1.67 |
| 2112-mp3 | TSK22014 | F01 | 205 | 18.25 | 1.74 | 2.12 |
| 2112-mp3 | TSK22014 | G01 | 206 | 12.13 | 1.78 | 1.48 |
| 2112-mp3 | TSK22014 | H01 | 208 | 38.04 | 1.63 | 0.83 |
| 2112-mp3 | TSK22014 | A02 | 211 | 16.47 | 1.79 | 1.72 |
| 2112-mp3 | TSK22014 | B02 | 213 | 15.74 | 1.77 | 1.78 |
| 2112-mp3 | TSK22014 | C02 | 216 | 14.37 | 1.72 | 1.74 |
| 2112-mp3 | TSK22014 | D02 | 220 | 15.79 | 1.73 | 1.45 |
| 2112-mp3 | TSK22014 | E02 | 221 | 13.49 | 1.65 | 1.42 |
| 2112-mp3 | TSK22014 | F02 | 222 | 15.09 | 1.69 | 1.52 |
| 2112-mp3 | TSK22014 | G02 | 223 | 18.85 | 1.76 | 2.13 |
| 2112-mp3 | TSK22014 | H02 | 224 | 20.09 | 1.74 | 1.94 |
| 2112-mp3 | TSK22014 | A03 | 230 | 12.77 | 1.78 | 1.69 |
| 2112-mp3 | TSK22014 | B03 | 231 | 11.94 | 1.71 | 1.65 |
| 2112-mp3 | TSK22014 | C03 | 232 | 11.37 | 1.57 | 1.15 |
| 2112-mp3 | TSK22014 | D03 | 233 | 17.60 | 1.69 | 1.70 |
| 2112-mp3 | TSK22014 | E03 | 193 | 12.14 | 1.70 | 1.78 |
| 2112-mp3 | TSK22014 | F03 | 194 | 13.39 | 1.68 | 2.01 |
| 2112-mp3 | TSK22014 | G03 | 195 | 12.97 | 1.73 | 1.76 |
| 2112-mp3 | TSK22014 | H03 | 197 | 18.99 | 1.75 | 2.06 |
| 2112-mp3 | TSK22014 | A04 | 198 | 16.80 | 1.71 | 1.49 |
| 2112-mp3 | TSK22014 | B04 | 200 | 11.74 | 1.74 | 2.20 |
| 2112-mp3 | TSK22014 | C04 | 203 | 17.80 | 1.72 | 1.67 |
| 2112-mp3 | TSK22014 | D04 | 207 | 12.67 | 1.68 | 2.56 |
| 2112-mp3 | TSK22014 | E04 | 209 | 16.76 | 1.73 | 1.87 |
| 2112-mp3 | TSK22014 | F04 | 210 | 14.48 | 1.53 | 1.34 |
| 2112-mp3 | TSK22014 | G04 | 212 | 11.67 | 1.70 | 1.40 |
| 2112-mp3 | TSK22014 | H04 | 214 | 10.27 | 1.72 | 2.31 |
| 2112-mp3 | TSK22014 | A05 | 215 | 17.04 | 1.70 | 1.73 |
| 2112-mp3 | TSK22014 | B05 | 217 | 18.87 | 1.74 | 1.80 |
| 2112-mp3 | TSK22014 | C05 | 218 | 15.95 | 1.72 | 1.66 |
| 2112-mp3 | TSK22014 | D05 | 219 | 19.43 | 1.57 | 1.22 |
| 2112-mp3 | TSK22014 | E05 | 225 | 15.25 | 1.65 | 1.21 |
| 2112-mp3 | TSK22014 | F05 | 226 | 15.54 | 1.73 | 1.83 |
| 2112-mp3 | TSK22014 | G05 | 227 | 11.22 | 1.55 | 1.30 |
| 2112-mp3 | TSK22014 | H05 | 228 | 10.02 | 1.70 | 2.42 |
| 2112-mp3 | TSK22014 | A06 | 229 | 9.63 | 1.72 | 2.32 |
| 2112-mp3 | TSK22014 | B06 | 234 | 7.66 | 1.63 | 3.84 |
| 2112-mp3 | TSK22014 | C06 | 235 | 16.00 | 1.54 | 0.97 |
| 2112-mp3 | TSK22014 | D06 | 236 | 22.68 | 1.65 | 1.38 |
| 2112-mp3 | TSK22014 | E06 | 237 | 14.24 | 1.67 | 1.77 |
| 2112-mp3 | TSK22014 | F06 | 9612-1 | 19.22 | 1.73 | 1.96 |
| 2112-mp3 | TSK22014 | G06 | 9612-2 | 20.15 | 1.77 | 1.73 |
| 2112-mp3 | TSK22014 | H06 | 9612-3 | 32.94 | 1.78 | 1.81 |
| 2112-mp3 | TSK22014 | A07 | 251 | 22.28 | 1.80 | 2.01 |
| 2112-mp3 | TSK22014 | B07 | 252 | 12.88 | 1.65 | 1.40 |
| 2112-mp3 | TSK22014 | C07 | 253 | 20.83 | 1.76 | 1.96 |
| 2112-mp3 | TSK22014 | D07 | 254 | 18.95 | 1.73 | 1.80 |
| 2112-mp3 | TSK22014 | E07 | 255 | 10.92 | 1.68 | 1.55 |
| 2112-mp3 | TSK22014 | F07 | 256 | 14.87 | 1.73 | 1.82 |
| 2112-mp3 | TSK22014 | G07 | 257 | 17.91 | 1.78 | 1.66 |
| 2112-mp3 | TSK22014 | H07 | 258 | 18.05 | 1.74 | 1.56 |
| 2112-mp3 | TSK22014 | A08 | 259 | 22.61 | 1.81 | 2.41 |
| 2112-mp3 | TSK22014 | B08 | 260 | 15.25 | 1.72 | 1.67 |
| 2112-mp3 | TSK22014 | C08 | 261 | 19.79 | 1.75 | 1.42 |
| 2112-mp3 | TSK22014 | D08 | 262 | 35.61 | 1.75 | 1.89 |
| 2112-mp3 | TSK22014 | E08 | 263 | 46.79 | 1.79 | 2.07 |
| 2112-mp3 | TSK22014 | F08 | 264 | 21.71 | 1.76 | 1.65 |
| 2112-mp3 | TSK22014 | G08 | 265 | 20.31 | 1.78 | 1.53 |
| 2112-mp3 | TSK22014 | H08 | 266 | 17.95 | 1.78 | 1.74 |
| 2112-mp3 | TSK22014 | A09 | 267 | 25.73 | 1.79 | 2.19 |
| 2112-mp3 | TSK22014 | B09 | 268 | 25.14 | 1.76 | 1.80 |
| 2112-mp3 | TSK22014 | C09 | 9405-1 | 20.23 | 1.78 | 2.50 |
| 2112-mp3 | TSK22014 | D09 | 9405-2 | 26.21 | 1.76 | 1.80 |
| 2112-mp3 | TSK22014 | E09 | 9405-3 | 25.44 | 1.75 | 1.92 |
| 2112-mp3 | TSK22014 | F09 | 390-1 | 29.49 | 1.77 | 1.66 |
| 2112-mp3 | TSK22014 | G09 | 390-2 | 26.13 | 1.76 | 1.73 |
| 2112-mp3 | TSK22014 | H09 | 390-3 | 27.10 | 1.78 | 1.88 |
| 2112-mp3 | TSK22014 | A10 | 9302-1 | 21.02 | 1.80 | 2.29 |
| 2112-mp3 | TSK22014 | B10 | 9302-2 | 19.97 | 1.80 | 2.93 |
| 2112-mp3 | TSK22014 | C10 | 9302-3 | 24.89 | 1.79 | 1.80 |
| 2112-mp3 | TSK22014 | D10 | 8150-1 | 20.10 | 1.79 | 2.33 |
| 2112-mp3 | TSK22014 | E10 | 8150-2 | 29.77 | 1.77 | 1.76 |
| 2112-mp3 | TSK22014 | F10 | 8150-3 | 19.73 | 1.77 | 1.84 |
| 2112-mp3 | TSK22014 | G10 | 9388-1 | 51.09 | 1.76 | 1.53 |
| 2112-mp3 | TSK22014 | H10 | 9388-2 | 34.90 | 1.76 | 1.65 |
| 2112-mp3 | TSK22014 | A11 | 9388-3 | 33.40 | 1.80 | 2.17 |
| 2112-mp3 | TSK22014 | B11 | 9709-1 | 16.90 | 1.78 | 2.86 |
| 2112-mp3 | TSK22014 | C11 | 9709-2 | 29.45 | 1.75 | 1.90 |
| 2112-mp3 | TSK22014 | D11 | 9709-3 | 19.93 | 1.77 | 1.95 |
| 2112-mp3 | TSK22014 | E11 | 1441-1 | 22.07 | 1.80 | 2.79 |
| 2112-mp3 | TSK22014 | F11 | 1441-2 | 17.46 | 1.78 | 2.58 |
| 2112-mp3 | TSK22014 | G11 | 1441-3 | 13.45 | 1.74 | 2.56 |
| 2112-mp3 | TSK22014 | H11 | 9705-1 | 25.46 | 1.75 | 2.54 |
| 2112-mp3 | TSK22014 | A12 | 9705-2 | 22.14 | 1.81 | 2.78 |
| 2112-mp3 | TSK22014 | B12 | 9705-3 | 27.71 | 1.77 | 2.81 |
| 2112-mp3 | TSK22014 | C12 | 9450-1 | 22.59 | 1.79 | 2.71 |
| 2112-mp3 | TSK22014 | D12 | 9450-2 | 21.87 | 1.80 | 2.57 |
| 2112-mp3 | TSK22014 | E12 | 9450-3 | 23.42 | 1.79 | 2.60 |
| 2112-mp3 | TSK22014 | F12 | — | — | — | — |
| 2112-mp3 | TSK22014 | G12 | — | — | — | — |
| 2112-mp3 | TSK22014 | H12 | — | — | — | — |

The phenotypes of the mapping populations (see Table 6 and Table 7, respectively) were sent to NRGene Ltd. for the mapping analyses.

### ToBRFV Resistance Phenotype Characterization

The inoculum used is the one isolated and maintained by Dr. Aviv Dombrovsky from the Agricultural Research Organization, Volcani Center, Israel. An inoculation of the seedlings conducted at 1-2 real leaves stage and the evaluation of resistance was done after 3-4 weeks as described at Avner Zinger et. al., Plants 2021, 10, 179., 2021. The determination of resistance was carried out using the bioassay method which comprises: (i) lack of phenotypic symptoms after actively infecting the plants with the virus and (ii) no symptoms on Nicotiana Glutinosa plants that were infected with the infected plant's sap.

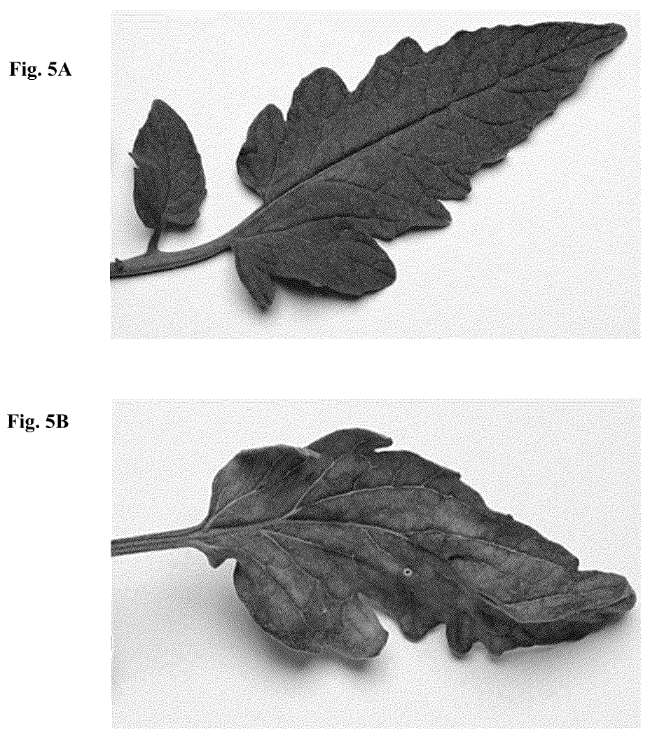

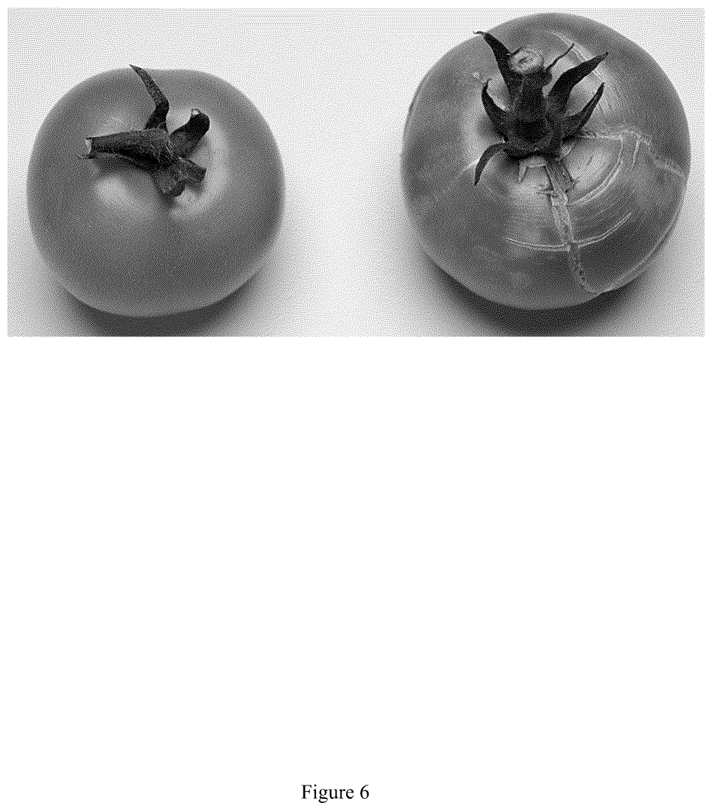

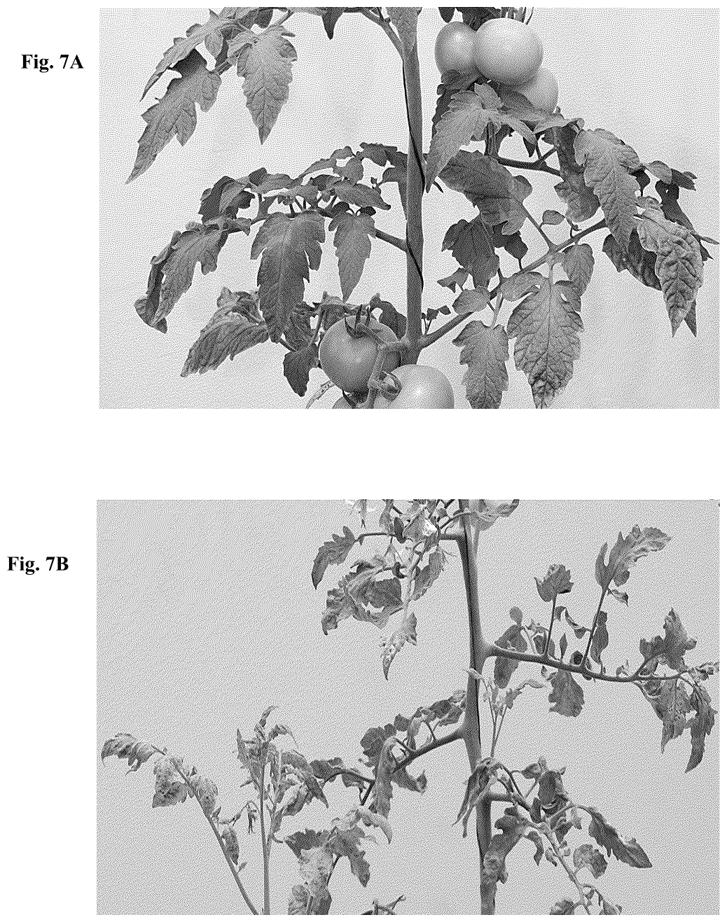

FIG. 5-7 depict the ToBRFV-resistant tomato plant of the present invention (generated according to the crossing programs above) compared to a sensitive tomato plant.

### EXAMPLE 4

To proceed with obtaining a ToBRFV-resistant cultivated tomato plant, wherein the resistance involves a partially dominant gene or genes, the following steps are taken:

Generating segregating populations such as F2 and F3 populations of the susceptible cultivated line x S. Habrochaites accession, and backcross populations, for QTL/gene mapping of this partially dominant resistant cross. The bioassays described in example 1 (monitoring symptoms after an infection and the Nicotiana glutinosa assay) are executed on all generations to confirm the presence of partially dominant resistance. Once the QTL or gene/genes conferring partially dominant resistance to ToBRFV are identified, they could be utilized as novel genetic markers. Those markers could be harnessed to rapidly identify and select ToBRFV-resistant tomato varieties/cultivars without the need to perform the above-mentioned bioassays, which are time-consuming and laborious.

In addition to testing resistant populations derived from crossing the susceptible cultivated line x S. Habrochaites accession, a further goal of this invention is to germinate F1 hybrids of susceptible cultivated lines crossed with the other 8 resistant wild species accessions, which are mentioned in example 2. The F1 hybrids are infected with ToBRFV and determined to see if they harbor resistance dominance, by monitoring symptoms and conducting genetic analyses such as QTL mapping to discover the partially dominant resistance gene or genes.

Another step of the present invention is producing germinating F1 hybrids of susceptible wild accessions lines crossed with resistant plants derived from the 9 accessions (LA0107, LA0361, LA1918, LA2650, LA1938, LA1969, LA2748, LA2755 and LA2931). After generating these crosses, the hybrid F1 plants are infected with ToBRFV and determined to see if they harbor resistance dominance. This mediating cross is designed to facilitate obtaining fertile F1 seeds that then could be crossed with S. lycopersicum cultivated lines.

An additional step of the present invention is crossing the resistant plants of the above 9 accessions with the F1 hybrid (disclosed in example 2) that already germinated and verified for being resistant. These types of crosses are destined to achieve two goals: (i) a bridging cross to overcome germination problems by creating a germinating 3-way cross; and (ii) investigating the resistance obtained by combinations of different resistance sources.

### EXAMPLE 5

### Tissue Collection, DNA Extraction and Sequencing

To evaluate and confer the genetic base of the partially dominant resistance of the obtained ToBRFV-resistant cultivated tomato plant resulting from all the steps taken as disclosed in example 4, plant tissues were collected, DNA was extracted sequenced for the two populations BC1F1-96/20-28 (population 1) and BC2F1-95/20-54 (population 2)

For the plant tissue collection, a BioArk leaf collection kits, were used (see Appendix 1). DNA extraction was performed by LGC Genomics Ltd (Hoddesdon, Herts EN11 OWZ, UK, www.lgcgroup.com/about-us/locations/). DNA sequencing was performed by Psomagen, Inc. (1330 Piccard Drive, Ste 103, Rockville, MD 20850, US).

### Population 1, BC1F1-96/20-28

Tissues were collected by the Philoseed into 4 BioArk Leaf collection kits (96 samples each) according to the manufacturer guidelines (see Appendix 1) and sent to LGC Genomics Ltd (Hoddesdon, Herts EN11 0WZ, UK) for DNA extraction service Plant tissue, supplied in 96 well plate—for Next Generation Sequencing (NGS)—1 to 4 plates.

The first plate was only parental lines tissues (collected in triplicates) of which the relevant parental lines for the mapping population were selected (see above Table 3).

The other three plates containing 237 samples of the resistant trait segregating population samples were sent to LGC Genomics Ltd for the same DNA extraction procedure as disclosed above. The tissue collection plate maps are depicted in the above Table 1.

All DNA extraction samples were tested for quality, and the samples DNA extraction quality was sufficient for library preparation and Skim-Sequencing (see above Table 4 and Table 5), All DNA samples were sent to Psomagen, Inc. for sequencing.

Upon arrival to Psomagen, Inc. DNA samples were Quality controlled (Qced) and found to meet the Sequencing quality requirements (see Appendix 2).

Parental lines triplicate DNA samples were pooled and QCed, Next TruSeq DNA PCR Free sequencing libraries (www.illumina.com/content/dam/illumina-marketing/documents/products/datasheets/datasheet_truseq_dna_per_free_sample_prep.pdf) were prepared.

Progeny DNA samples were QCed and iGenomX Riptide sequencing libraries (igenomx.com/how-riptide-works/) were prepared.

Parental lines libraries were sequenced on an Illumina NovaSeq 6000 sequencer with 150 bp paired end reads, to ˜x35 coverage calculated by a genome size of 0.9 Gbp.

Progeny samples libraries were sequenced on an Illumina NovaSeq 6000 sequencer with 150 bp paired end reads, to ˜x2 coverage calculated by a genome size of 0.9 Gbp.

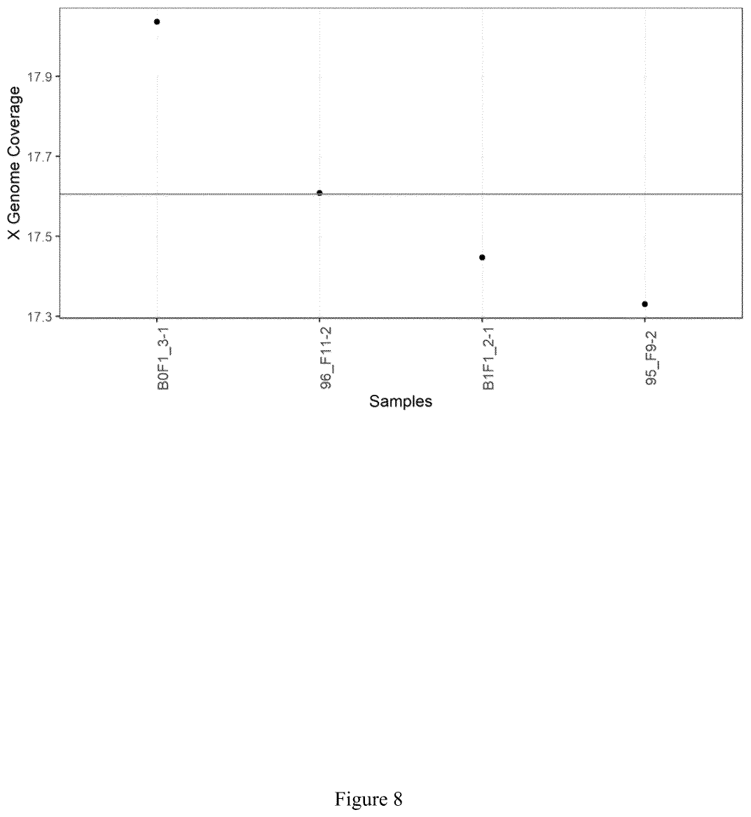

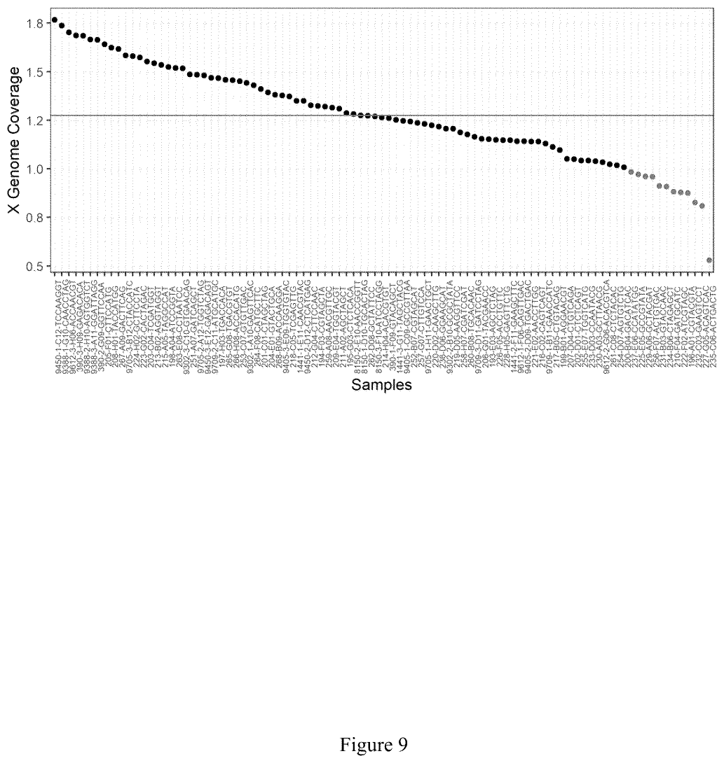

Sequencing data was uploaded to NRGenes AWS-S3 cloud storage. Reference is now made to FIG. 8-9 cross the two population and parental lines samples. sequencing data coverage FIG. 8 depicting the parental lines and FIG. 9 depicting population 1 20-28, more particularly, the 98 samples that were selected for analysis

To further estimate the quality of the raw data, the FastQC software has been run on the sequencing data and all sequencing libraries QC results were aggregated into MultiQC report. The quality indicators of the data as displayed by MultiQC have met with NRGene's standards requirements. The quality parameters of MultiQC results are available on the MultiQC website (see: www.multiqc.info).

### Population 2, BC2F1-95/20-54

161 Leaf tissues samples were collected by Philoseed in two 96 well plates and sent to Gene-G ltd, for DNA extraction (see Table 2).

All DNA extraction samples were tested for quality, and the samples DNA extraction quality was sufficient for library preparation and Skim-Sequencing. All DNA samples were sent to Psomagen, Inc. for sequencing.

Upon arrival to Psomagen, Inc. DNA samples were QCed and found to meet the Sequencing quality requirements (see Appendix 2).

Progeny DNA samples were QCed and iGenomX Riptide sequencing libraries (igenomx.com/how-riptide-works/) were prepared.

Progeny samples libraries were sequenced on an Illumina NovaSeq 6000 sequencer with 150 bp paired end reads, to ˜x2 coverage calculated by a genome size of 0.9 Gbp.

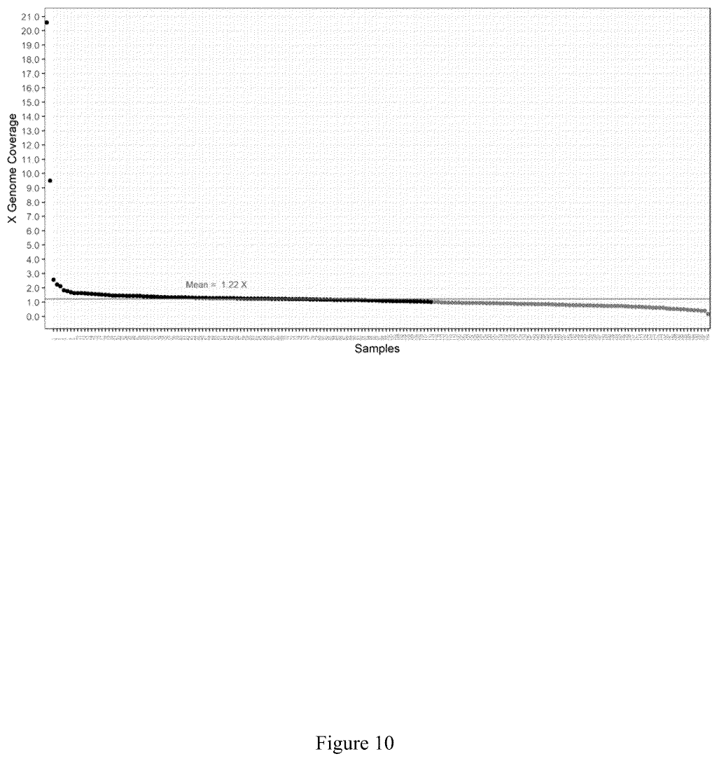

Sequencing data was uploaded to NRGenes AWS-S3 cloud storage. Reference is now made to FIG. 10, depicting population 2, sequencing data coverage cross samples.

### EXAMPLE 6

### Validation of the Bioassay Test

To further evaluate the appearance of the Tobamovirus at the plants of the mapping populations, the reliability of the bioassay (glutinosa test) was tested. Fresh leaves samples from 47 selected plants were sent for both tests (i.e. Nicotiana glutinosa and ELISA) to Microlab Laboratories LTD. The samples were tested for:
1. Tobamovirus (TMV, ToMV, ToBRFV)—by Bioassay method on Nicotina glutinosa test plants.
2. Tomato Brown Rugose Fruit Virus (ToBRFV)—by ELISA (Enzyme linked immunosorbent assay) method.

The tests were performed according to:
S.O.P 50: “Testing samples by the ELISA method”. ToBRFV: Bioreba, Lot: 150805/160805, Exp.: April 2023.
S.O.P 56: “Detection of infectious Tobamovirus in tomato and pepper plants and seeds” (Tomato: TMV, ToBRFV) (Pepper: TMV, ToMV, PMMoV, ToBRFV).

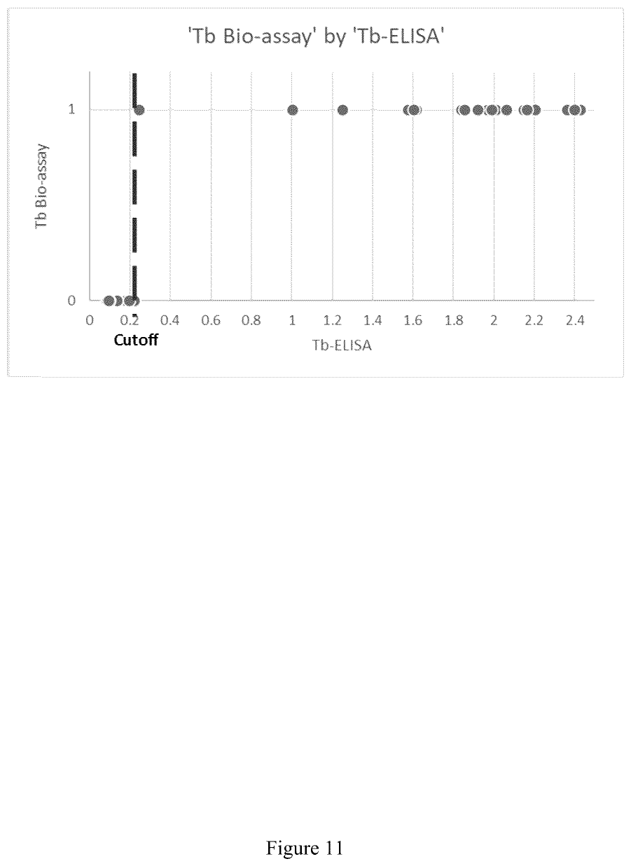

The samples for the ELISA were tested and analyzed with the appropriate controls (i.e. Blank, Positive control, Negative control), read on OD405 nm, and Cutoff value was determined. The test performed was under accreditation to ISO/IEC 17025 standard. Refernce is now made to Table 8 below and FIG. 11 depicting the correlation between ToBRFV ELISA test and Nicotina glutinosa Bioassay.

Following summarizing the results a complete correlation was found between the two tests, which support the bioassay we conducted as part of the mapping population plants phenotyping.

### EXAMPLE 7

### QTL Analysis

The phenotype data of the two mapping populations, Population 1 and 2 (BC1F1-96/20-28, BC2F1-95/20-54) details 3 resistance categories (see Table 6 and Table 7 above):
1. Susceptible plants (S)—Early age asymptomatic and positive at bioassay
2. Intermediate Resistant plants (IR)—None/late age asymptomatic and positive at bioassay (some of his group may be “s” but were not planted).
3. High Resistant plants (HR) Asymptomatic and negative at bioassay, this category is exhibiting a strong resistance that may be considered as immunity.

### Data Pre-Processing

The genomes of all parental lines (B0F1_3-1, 96_F11-2, B1F1_2-1, 95_F9-2) were assembled to a chromosome level using NRgene Assembly technology, using the Heinz genome assembly (version SL4.0) as reference. The reference genome used in the pipeline was downloaded from solgenomics.net/organism/Solanum_lycopersicum/genome (version SL4.0). Briefly, each genome was assembled to a contig level and the contigs were mapped to the reference genome based on sequence identity.

The assembled contigs were split into non-overlapping K-mers of 74 bp and a K-mer database (KDB) for each parental line was constructed holding all the corresponding K-mers.

The KDB for each line was filtered by comparing the K-mers between the different lines and keeping unique K-mers that appear exactly once in the corresponding line and none in the other lines.

This was done using an exact match comparison between any two K-mers. Each unique K-mer is then searched in the progeny to generate an absence/presence table for each K-mer and Progeny-sample.

We then aggregated the K-mer counts by contigs a Contig-level Count Matrix (CCM) was constructed with presence/absence values per sample in the progeny. The values in CCM were normalized by sample sequencing depth and the number of contig K-mers to generate Contig-level Genotyping Matrix (CGM) having presence/absence values for each sample on each contig.

This pipeline produces a CGM that represents contigs originating from the wild tomato accession genome since all other sequences from the cultivated lines were filtered out.

The number of contigs for each of the Back cross (BC) parents with a status of the mapping to the reference genome is depicted in Table 9 below.

TABLE 9
Number of contigs for each of the Back cross (BC) parents
with a status of the mapping to the reference genome

| Pop | Mapped | Count |
|---|---|---|
| Population-1 | FALSE | 11952 |
| | TRUE | 4719 |
| Population-2 | FALSE | 8298 |
| | TRUE | 4228 |

### QTL Analysis

QTL analysis was performed using a regression model on each of the contigs using the values stored in the CGM. This regression is related to the wild sample inheritance patterns as CGM represents the alleles originated from the wild sample only. QTL scans for the ToBRFV tolerance phenotype was conducted as follows. Table 10 depicts the phenotype categorical values and a corresponding score for each population. The number of samples in each category is indicated in the Count column for the respective population. The R-score was used as the phenotype value in the search for QTL.

TABLE 10
Phenotype categorical values and a corresponding
score for each population.

| Phenotype | Rscore | Count -Pop1 | Count -Pop2 |
|---|---|---|---|
| HR | m definition | 27 | 6 |
| IR | 1 | 22 | 37 |
| S | 0 | 45 | 118 |

a QTL scan was performed by regressing the phenotype score on the genotype at each contig. A significant QTL was declared if a model including the genotype was substantially better than a model without the genotype using a likelihood-ratio test.

A logarithm Of Odds (LOD) score was calculated by comparing the variance explained by the two models as indicated in Broman et al. (Ch 4.1, Pg 77). A Threshold of LOD>3 was used to declare significant results. Contigs that passed the LOD>3 threshold were subject to further examination and confidence intervals were calculated for the selected peaks based on 1.5 units in LOD scores from the most significant contig.

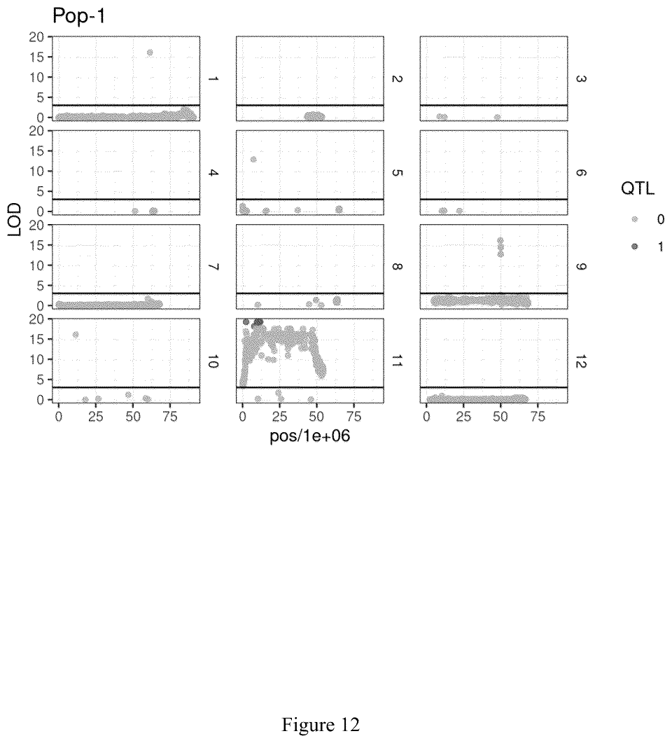

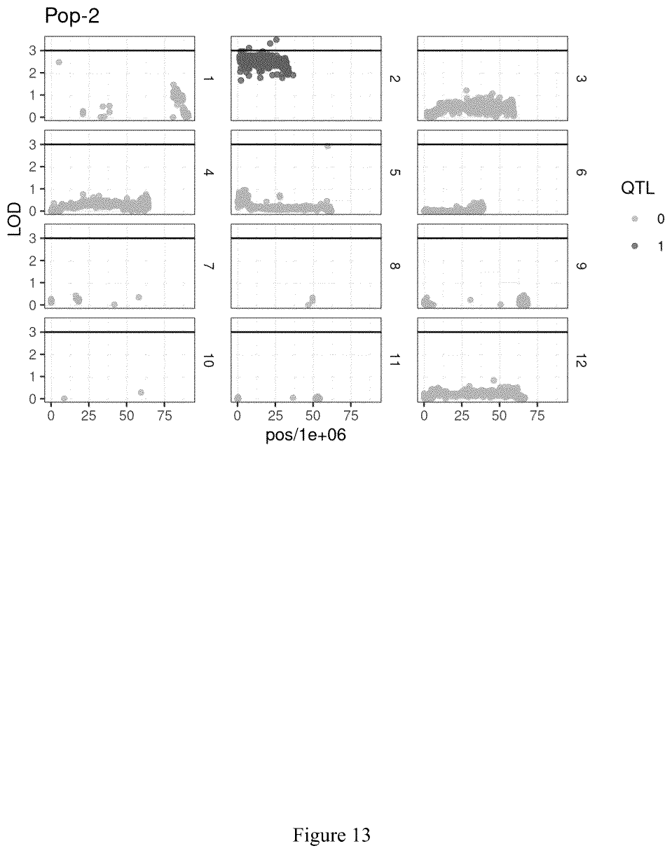

reference is now made to FIG. 12 and FIG. 13 depicting the LOD scores at each contig for each of the populations (pop-1 and pop-2). The panels describe the different chromosomes based on the reference. Only contigs that were successfully mapped to the reference genome are shown. A horizontal line indicating LOD=3 is indicated in black.

Significant regions in darker grey indicating the confidence interval of each peak. In both populations, contigs with an unmapped status were examined and all significant contigs were found to be correlated with the main peaks, which led to the conclusion that there are no alternative signals in the unmapped contigs.

### Population-1

Sequences from the wild accession were found to be present in a significant proportion on Chromosomes 1, 7, 9, 11 and 12. On Chromosome 11 a large and highly significant peak spans most of the Chromosome. At the peak the explained phenotypic variance is 60%. There is evidence in the LOD score curve that the source of this correlation is at the beginning of the Chromosome. The 95% confidence interval for the suggested region is at: Chr11:2-12 [Mbp]. All other significant contigs that are found on other chromosomes were found to be correlated with the peak on chromosome 11 indicating a difference in the physical structure of the reference genome (solgenomics.net/organism/Solanum_lycopersicum/genome (version SL4.0) and the current BC parent, in other words, the physical mapping in these cases is incorrect).

### Population-2

Sequences from the wild accession were found to be present in a significant proportion on Chromosomes 2, 3, 4, 5, 6 and 12. On Chromosome 2 a large peak which spans the short arm is present and a few contigs pass the LOD>3 threshold. This suggestive region is the only part in the genome which is significant at an False Discovery Rates (FDR)<0.05 and spans a confidence interval at Chr2:1-37 [Mbp]. The relatively weak signal in this population as compared to signal in population 1, is probably related to the small number of HR samples.

### Genotype/Phenotype Counts

The effect of each genotype on the phenotype at the peak of the QTL loci is depicted in Table 11. The population column indicates the corresponding pedigree. The Genotypes column indicates the presence/absence of a selected contig at the peak of each QTL: 0 indicates absence and 1 indicates presence. The columns HR, IR and S hold the number of samples found in each category. The table suggests that for QTL1 (Chr11) the presence of the wild allele conveys sensitivity with an odds ratio of 42:3. For QTL2 the presence of the wild allele conveys resistance although odds ratios could not be calculated due to the small sample size.

TABLE 11
Effect of each genotype on the phenotype
at the peak of the QTL loci

| Population | Chr | Genotype | HR | IR | S |
|---|---|---|---|---|---|
| Pop1 (QTL1) | 11 | 0 | 26 | 14 | 3 |
| | | 1 | 1 | 8 | 42 |
| Pop2 (QTL2) | 2 | 0 | 0 | 12 | 71 |
| | | 1 | 6 | 25 | 47 |

### EXAMPLE 8

### Marker Sequences

Unique sequences that could be used as molecular markers for the selected regions were extracted using the following pipeline:
1. Extract K-mers of 1000 bp from the BC samples.
2. Filter for poor GC content (keep sequences with 30<GC<70).
3. Filter using BLAST against the cultivated reference (solgenomics.net/organism/Solanum_lycopersicum/genome version SL4.0) by requiring an alignment proportion smaller than 40% within each K-mer.

1000 bp sequences markers derived from the wild accession sequences for each of the chromosomal loci that showed correlation with the phenotype were identified (see Appendix 3):
1. For chromosome 2 there are 26 sequences spanning over a region of ˜48 Mbp
2. For chromosome 11 there are 8 sequences spanning over a region of ˜3 Mbp

As disclosed above the Chromosome 2 region is correlated with the resistance phenotype while the chromosome 11 region is correlated with the sensitivity phenotype. Hence, in order to predict resistant plants, markers at chr-2 should be selected for and markers at chr-11 should be selected against.

1.-31. (canceled)

32. A tomato plant harboring a partially dominant resistance gene or genes to tomato brown rugose fruit virus (ToBRFV) wherein said plant is the offspring of crossing a ToBRFV-resistant wild tomato plant with a ToBRFV-susceptible cultivated tomato plant or a TOBRFV-resistant wild tomato plant with a ToBRFV-susceptible wild tomato plant; and
wherein, said ToBRFV-resistant wild tomato plant harbors said partially dominant resistance gene or genes and said ToBRFV-resistant wild tomato plant is selected from a group consisting of LA0107 (S. corneliomulleri), LA0361, LA1918, LA2650 (S. habrochaites), LA1938, LA1969, LA2748, LA2755 and LA2931 (S. chilense); and
further wherein said ToBRFV-susceptible cultivated tomato plant is Solanum lycopersicum

33. A seed of the tomato plant of claim 32.

34. Propagation materials of the tomato plant of claim 32.

35. Genetic markers for the use in identification or selection of tomato plants harboring partially dominant resistance to tomato brown rugose fruit virus (ToBRFV),
wherein said markers are configured to be linked to the gene or genes or QTL conferring partially dominant resistance to ToBRFV in said tomato plants;

36. The genetic markers according to claim 35, wherein said gene or genes or QTL is on chromosome 2; and
wherein said gene, or genes, or QTL on chromosome 2 comprises at least one genomic sequences sequence selected from a group under key number and position as indicated in Appendix 3:

| | | |
|---|---|---|
| BC1_2_L2_15031000 | 15031000, BC1_2_L2_18820000 | 18820000, |
| BC1_2_L2_15795000 | 15795000, BC1_2_L3_29720000 | 29720000, |
| BC1_2_L3_22563000 | 22563000, BC1_2_L3_25275000 | 25275000, |
| BC1_2_L3_25187000 | 25187000, BC1_2_L3_29656000 | 29656000, |
| BC1_2_L3_24977000 | 24977000, BC1_2_L3_24955000 | 24955000, |
| BC1_2_L3_27949000 | 27949000, BC1_2_L3_23415000 | 23415000, |
| BC1_2_L3_28862000 | 28862000, BC1_2_L3_26512000 | 26512000, |
| BC1_2_L3_24581000 | 24581000, BC1_2_L4_32405000 | 32405000, |
| BC1_2_L4_33723000 | 33723000, BC1_2_L4_33383000 | 33383000, |
| BC1_2_L4_32180000 | 32180000, BC1_2_L4_33110000 | 33110000, |
| BC1_2_L4_32404000 | 32404000, BC1_2_L4_34990000 | 34990000, |
| BC1_2_L4_35114000 | 35114000, BC1_2_L4_33583000 | 33583000, |
| BC1_2_L4_35860000 | 35860000, BC1_2_L4_32796000 | 32796000. |

37. Genetic markers for the use in identification and/or negative selection of tomato plants harboring sensitivity to tomato brown rugose fruit virus (ToBRFV)
wherein said markers are configured to be linked to the gene or genes or QTL conferring sensitivity to ToBRFV in said tomato plants.

38. The genetic markers according to claim 37, wherein said gene or genes or QTL is on chromosome 11; and
wherein said gene or genes or QTL on chromosome 11 comprises at least one genomic sequences sequence selected from a group under key number and position as indicated in Appendix 3:

| | | |
|---|---|---|
| BC0_11_L1_10826000 | 10826000, BC0_11_L1_9649000 | 9649000, |
| BC0_11_L1_10837000 | 10837000, BC0_11_L1_10827000 | 10827000, |
| BC0_11_L1_10141000 | 10141000, BC0_11_L2_11459000 | 11459000, |
| BC0_11_L2_12276000 | 12276000, BC0_11_L2_12706000 | 12706000. |

39. A method for identifying a tomato plant, or tomato seed, or tomato plant tissue, or tomato plant cell, harboring a partially dominant resistance gene or genes to tomato brown rugose fruit virus (ToBRFV) comprising steps:
a. obtaining a tomato plant, or tomato seed, or tomato plant tissue, or tomato plant cell,
b. identifying said genetic markers according to claim 35.

40. A method for identifying and/or selecting a tomato plant, or tomato seed, or tomato plant tissue, or tomato plant cell, harboring a sensitivity gene or genes to tomato brown rugose fruit virus (ToBRFV) comprising steps:
a. obtaining a tomato plant, or tomato seed, or tomato plant tissue, or tomato plant cell,
b. identifying said genetic markers according to claim 37.

41. A method for identifying and/or selecting a tomato plant, or tomato seed, or tomato plant tissue, or tomato plant cell, harboring a partially dominant resistance gene or genes or QTL to tomato brown rugose fruit virus (ToBRFV), and for identifying and/or selecting against a sensitivity gene or genes or QTL to tomato brown rugose fruit virus (ToBRFV) comprising steps:
a. obtaining a tomato plant, or tomato seed, or tomato plant tissue, or tomato plant cell,
b. identifying said genetic markers according to claim 35,
c. selecting the tomato plant, or tomato seed, or tomato plant tissue, or tomato plant cell, harboring said genetic markers,
d. identifying said genetic markers in the selected plants of section c,
e. selecting against the tomato plant, or tomato seed, or tomato plant tissue, or tomato plant cell, harboring said genetic markers,
f. obtaining a tomato plant, or tomato seed, or tomato plant tissue, or tomato plant cell harboring a partially dominant resistance gene or genes, or QTL, and lacking a sensitivity gene or genes, or QTL to tomato brown rugose fruit virus (ToBRFV).

42. A tomato plant harboring a partially dominant resistance gene or genes and lacking sensitivity to tomato brown rugose fruit virus (ToBRFV), wherein said plant is the offspring of crossing a ToBRFV-resistant wild tomato plant, which also lack sensitivity to ToBRFV, with a ToBRFV-susceptible cultivated tomato plant, or a TOBRFV-resistant wild tomato plant with a ToBRFV-susceptible wild tomato plant; and wherein
said ToBRFV-resistant wild tomato plant harbors said partially dominant resistance gene or genes and lacks said sensitivity genes; and wherein
said partially dominant resistance gene or genes is on chromosome 2, said sensitivity gene or genes is on chromosome 11.

43. Genetic markers for the use in identification or selection of tomato plants harboring partially dominant resistance and lacking sensitivity to tomato brown rugose fruit virus (ToBRFV), wherein said markers are configured to be linked to the gene or genes or QTL conferring partially dominant resistance to ToBRFV and sensitivity in said tomato plants.

44. The genetic markers according to claim 43, wherein said gene or genes or QTL conferring partially dominant resistance to ToBRFV is on chromosome 2 and wherein said gene or genes or QTL conferring sensitivity to ToBRFV is on chromosome 11.

45. The genetic markers according to claim 43, wherein said gene, or genes, or QTL conferring partially dominant resistance to ToBRFV on chromosome 2 comprises at least one genomic sequences sequence selected from a group under key number and position as indicated in Appendix 3:

| | | |
|---|---|---|
| BC1_2_L2_15031000 | 15031000, BC1_2_L2_18820000 | 18820000, |
| BC1_2_L2_15795000 | 15795000, BC1_2_L3_29720000 | 29720000, |
| BC1_2_L3_22563000 | 22563000, BC1_2_L3_25275000 | 25275000, |
| BC1_2_L3_25187000 | 25187000, BC1_2_L3_29656000 | 29656000, |
| BC1_2_L3_24977000 | 24977000, BC1_2_L3_24955000 | 24955000, |
| BC1_2_L3_27949000 | 27949000, BC1_2_L3_23415000 | 23415000, |
| BC1_2_L3_28862000 | 28862000, BC1_2_L3_26512000 | 26512000, |
| BC1_2_L3_24581000 | 24581000, BC1_2_L4_32405000 | 32405000, |
| BC1_2_L4_33723000 | 33723000, BC1_2_L4_33383000 | 33383000, |
| BC1_2_L4_32180000 | 32180000, BC1_2_L4_33110000 | 33110000, |
| BC1_2_L4_32404000 | 32404000, BC1_2_L4_34990000 | 34990000, |
| BC1_2_L4_35114000 | 35114000, BC1_2_L4_33583000 | 33583000, |
| BC1_2_L4_35860000 | 35860000, BC1_2_L4_32796000 | 32796000; |

and wherein said gene, or genes, or QTL conferring sensitivity to ToBRFV on chromosome 11 comprises at least one genomic sequences sequence selected from a group under key number and position as indicated in Appendix 3:

| | | |
|---|---|---|
| BC0_11_L1_10826000 | 10826000, BC0_11_L1_9649000 | 9649000, |
| BC0_11_L1_10837000 | 10837000, BC0_11_L1_10827000 | 10827000, |
| BC0_11_L1_10141000 | 10141000, BC0_11_L2_11459000 | 11459000, |
| BC0_11_L2_12276000 | 12276000, BC0_11_L2_12706000 | 12706000. |

46. The tomato plant of claim 42, wherein said ToBRFV-resistant wild tomato plant, which also lack sensitivity to ToBRFV, is selected from a group consisting of LA0107 (S. corneliomulleri), LA0361, LA1918, LA2650 (S. habrochaites), LA1938, LA1969, LA2748, LA2755 and LA2931 (S. chilense).

47. The tomato plant of claim 42, wherein said ToBRFV-susceptible cultivated tomato plant is Solanum lycopersicum.

48. A seed of the tomato plant of claim 42.

49. Propagation materials of the tomato plant of claim 42.
